(19) United States
(12) Patent Application Publication
(10) Pub. No.: US 2009/0005726 A1
(43) Pub. Date: Jan. 1, 2009

(54) Insulin Pump Having Expected Bolus Interval and an Early Bolus Interval

(76) Inventors: Steven Paul Jones, Rochester, MN (US); Michael Launsbach, Rochester, MN (US)

Correspondence Address: Robert R. Williams;IBM Corporation, Dept. 917, 3605 Highway 52 North, Rochester, MN, 55901-7829, US

(21) Appl. No.: 11/769,401

(22) Filed: Jun. 27, 2007

(51) Int. Cl. A61M 5/168; G06F 17/00

(52) U.S. Cl. 604/65; 700/90

(57) ABSTRACT

An insulin pump having an expected bolus interval and an early bolus interval. The early bolus interval immediately precedes the expected bolus interval. The insulin pump alerts the user if no bolus is taken during an expected bolus interval unless a bolus was taken during the early bolus interval. The insulin pump queries the user if the bolus taken during the early bolus interval is to be interpreted as satisfying the expected bolus interval; if so, no alert is given if no bolus is then taken during the expected bolus interval; if not, an alert is given if no bolus is taken during the expected bolus interval.

### BACKGROUND OF THE INVENTION

1. Field of the Invention

This invention relates generally to insulin pumps. More particularly, the present invention relates to insulin pumps configured to alert users when a bolus is not taken during an interval during which a bolus is expected to be taken.

2. Description of the Related Art

People require insulin to survive. A non-diabetic person produces insulin in his or her pancreas in proper amounts so that the non-diabetic person's blood glucose concentration remains in a non-diabetic range. A type-1 diabetic makes little or no insulin in his or her pancreas and requires insulin (or insulin analogs, which are hereinafter to be included as “insulin”) to be brought into his or her body by way of syringe injections, CSII (Continuous Subcutaneous Insulin Infusion, also known as using an insulin pump), or inhaling insulin. A type-2 diabetic may produce too little insulin and may have insulin resistance, a condition in which insulin is poorly used by the body.

Many diabetics (primarily type-1 but some type-2) use an insulin pump in a CSII regimen. Insulin pump users take a bolus associated with a meal based primarily on how many grams of carbohydrates the meal contains. For example, if the meal contains one hundred grams of carbohydrates, and a particular diabetic requires one unit of insulin for every ten grams of carbohydrates, the diabetic would need to cause his or her insulin pump to pump ten units of insulin into his body. There is a wide range of insulin sensitivity among diabetics, and each diabetic must know how much insulin to take in a bolus for every gram of carbohydrate.

If the diabetic were to eat a meal containing carbohydrates and forget to take his or her bolus, the diabetic's blood sugar will rise to unhealthy levels, which is an undesirable condition.

Several patents are directed towards reminding a user of a forgotten meal bolus. For examples, U.S. Pat. No. 6,650,951 and U.S. Pat. No. 6,744,350 teach of a user programming an insulin pump with expected bolus intervals in which a bolus is expected to be taken, for example, on weekdays, a diabetic may normally have breakfast between 6:30 am and 7:30 am. Once programmed, the insulin pump will alert the diabetic if no bolus is taken between 6:30 am and 7:30 am. U.S. Pat. No. 6,999,854 teaches of an insulin pump that “learns” bolus patterns, thereby relieving the diabetic from having to program expected bolus intervals. At a high level, FIG. 2 describes the method 200 taught by the above patents. Method 200 begins at step 202. At step 204, an expected bolus interval is established (either by programming or by “learning”). In step 206, the insulin pump watches for an expected bolus interval to occur. The insulin pump has a built in timer (including day of the week, in some cases) and therefore knows a current time that is compared to start/end times (or start/duration times) of the expected bolus interval. If no bolus is taken during the expected bolus interval, as checked in step 208, the diabetic is alerted by vibration (or other tactile movement), by light, or by sound in step 210. Step 210 passes control to step 206. If step 208 determines that a bolus was taken during the expected bolus interval, control passes back to step 206.

There is a need for a method and apparatus for detecting if a bolus is taken within a predetermined time prior to a beginning of an expected bolus interval in order to prevent the diabetic from taking an unwanted second bolus responsive to being alerted that no bolus was taken during an expected bolus interval.

### SUMMARY OF THE INVENTION

The present invention provides method and apparatus embodiments for an insulin pump configured to provide a missed meal bolus alert.

The insulin pump has an expected bolus interval during which a bolus is expected to be taken by a user, and an early bolus interval that immediately precedes the expected bolus interval.

Embodiments of the invention are intended to prevent a situation where a diabetic user eats a first meal and takes a first bolus shortly before an expected bolus interval, then receives an alert that no bolus was taken during the expected bolus interval and takes a second bolus responsive to the alert. The second bolus, without eating a second meal is an extremely dangerous situation that could result in very serious consequences to the diabetic, including hospitalization or death. Since expected bolus intervals are typically an hour or more in duration, it is easy for the diabetic user to forget that he or she had already taken the first bolus.

In an embodiment, if no bolus is taken during the expected bolus interval, an alert will be issued unless a first bolus is taken during the early bolus interval and the first bolus is confirmed by the user as satisfying the expected bolus interval. If the user denies that the first bolus satisfies the expected bolus interval, the insulin pump will issue an alert at the end of the expected bolus interval unless a second bolus is taken during the expected bolus interval.

In an embodiment, if a bolus is taken during the early bolus interval, the user is asked if the associated expected bolus interval's start time should be changed to the time of the first bolus. If the user agrees, the start time of the expected bolus interval is moved to the time of the first bolus; if the user disagrees, the expected bolus interval is not changed.

### BRIEF DESCRIPTION OF THE DRAWINGS

FIGS. 1a and 1b show an insulin pump and a block diagram of components of the insulin pump.

FIG. 1c is a graphical depiction of three expected bolus intervals and their associated early bolus intervals versus time.

FIG. 2 shows a flow diagram of prior art missed bolus alert methods.

FIG. 3 illustrates a flowchart of a method embodiment of the invention.

FIG. 4 shows additional detail of a step in the method embodiment shown in FIG. 3.

FIG. 5 shows additional detail of a step in the method embodiment shown in FIG. 3.

FIG. 6 shows additional detail of a step in the method embodiment shown in FIG. 3.

FIG. 7 shows a flow chart of an alternative method embodiment of the invention.

### DETAILED DESCRIPTION OF THE PREFERRED EMBODIMENTS

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

Some modern insulin pumps have one or more instances of an expected bolus interval. An expected bolus interval is, in various embodiments, programmed into the insulin pump by a user, or is “learned” by the pump itself from bolus patterns observed by the pump.

It is possible that a user eats a first meal (and takes a first bolus for the first meal) shortly before a beginning of an expected bolus interval, and does not eat a second meal (and therefore does not take a second bolus) during the expected bolus interval. A conventional insulin pump will alert the user that no bolus was taken during the expected bolus interval. The user may take a second bolus in response to the alert, forgetting about the first bolus. The second bolus, in absence of the second meal, will cause the user's blood glucose to drop dangerously. If the user tests his or her blood sugar frequently, he or she may detect a low blood glucose condition and quickly eat enough carbohydrates as appropriate for the size of the second bolus. If the user does not test his or her blood sugar frequently, and has hypoglycemic unawareness (that is, he or she can not sense a “low blood sugar” (hypoglycemia) condition, the person may die.

Several exemplary variations of a method are presented which remind the user in the case where the user has taken a bolus within a predetermined time prior to a beginning of an expected bolus interval and ask the user if he or she really should take a second bolus if no bolus is taken during the expected bolus interval. It is possible that the user ate a first meal and bolused just prior to the expected bolus interval, and then ate a second meal but did not bolus during the expected bolus interval, in which case the second bolus would be needed, and one or more embodiments of the invention account for this possibility.

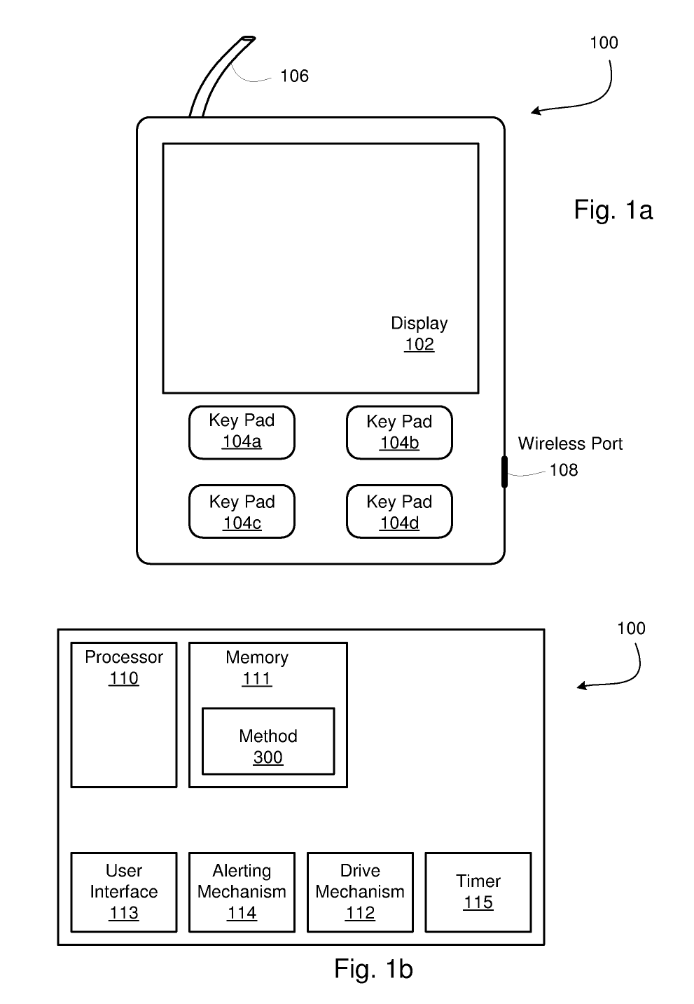

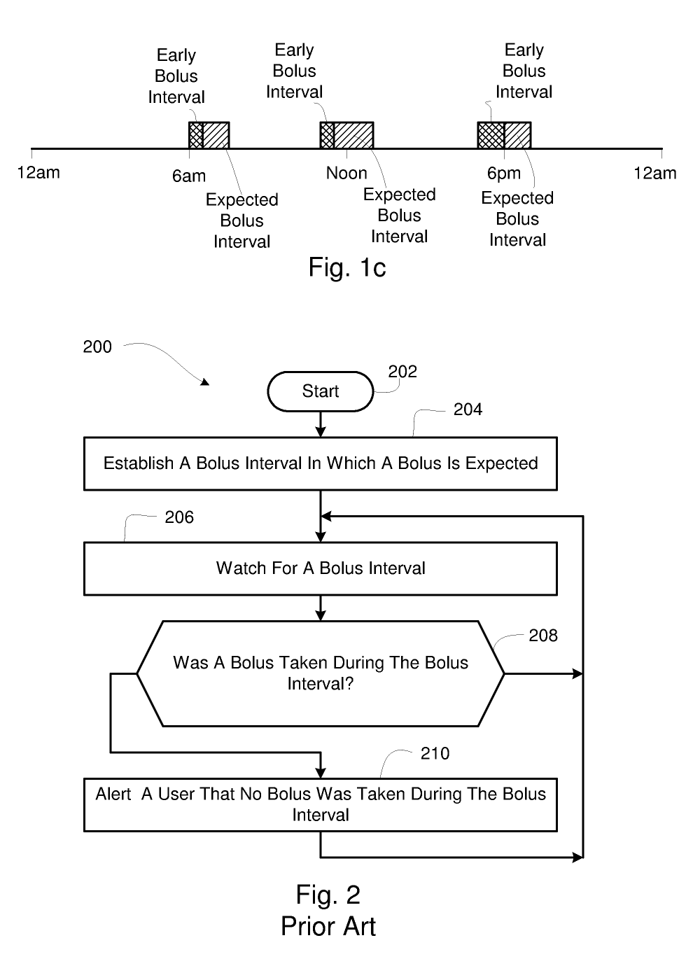

Turning now to FIG. 1a and FIG. 1b, an insulin pump having one or more embodiments of the invention is depicted.

FIG. 1a shows an insulin pump 100, having one or more embodiments of the invention, as seen by a user looking at insulin pump 100. Insulin pump 100 comprises a display 102 for viewing messages and prompts produced by the pump, and for providing the user feedback on information entered by the user. For example, insulin pump 100 may display a history of the user's recent boluses, information relating to taking a bolus during an early bolus interval (to be described later), and the like. Insulin pump 102 has one or more key pads 104 (104a-104d shown) by which the user enters information into the pump. Such information may include setting time of day, day of week, magnitude of a bolus, duration of bolus, and programming basal rate(s). Modern pumps have many values programmable by way of key pads.

Tubing 106 is coupled to an insulin reservoir (not shown) inside the insulin pump. Tubing 106 is not part of insulin pump 100 as such, but is shown to help understand that some means is needed to transport insulin from the insulin pump to the user.

Wireless port 108 can be an infrared port, a Bluetooth port, or the like, to provide communications with a computer. Many insulin pumps can be programmed by way of a wireless port, allowing easy programming using a GUI (graphical user interface) on a computer, followed by wireless transmission to the insulin pump, without having to use keypads 104.

FIG. 1b shows a block diagram of insulin pump 100. Insulin pump 100 comprises a processor 110 suitable for controlling operations of insulin pump 100. Processor 110 is coupled to a memory 111. Memory 111 stores information needed by processor 110. Typically at least a portion of memory 111 is implemented with nonvolatile storage so that programs and data are not lost during a power loss of a power source used to power electronic portions of insulin pump 100. For example, a power loss occurs during change of a battery (not shown). Nonvolatile storage includes an implementation wherein a normally volatile storage is temporarily kept powered by energy stored in a capacitor. Memory 111 further comprises computer program(s) and data that implement an embodiment of method 300 (described later).

Insulin pump 100 comprises one or more user interfaces 113 to interact with the user. Keypads 104, described earlier, are a user interface. Wireless port 108 provides another user interface via a computer. A wired interface is also contemplated but not shown.

Insulin pump 100 further comprises an alerting mechanism 114. Alerting mechanism 114 may produce audible sounds. Alerting mechanism 114 may produce a vibration or other tactile action suitable for sensing by touch by the user. Alerting mechanism 114 may produce a visual alert to the user, for example by flashing a light source (light emitting diode, for example). Alerting mechanism may produce some combination of audible, tactile, and visual alerts to the user.

Insulin pump 100 further comprises drive mechanism 112. Signals from processor 110 cause drive mechanism 112 to infuse insulin into the user. Typically, drive mechanism 112 further comprises a drive rod (not shown) driven by a motor (not shown), the drive rod pushing on a piston (not shown) in an insulin reservoir (not shown) such that, responsive to signals from processor 110, insulin is pushed from the reservoir, through tubing 106 (FIG. 1a) and into the user.

Insulin pump 100 further comprises a timer 115. Timer 115 keeps track of time of day and day of week. Timer 115 provides time information to processor 110, which then compares when a bolus is taken versus an expected bolus interval and/or an early bolus interval as will be described later.

It will be understood that modern insulin pumps may physically separate various elements. For example, a modern insulin pump may have a first physical unit containing the processor 110, memory 111, and user interface 113. Timer 115, alerting mechanism 114, and drive mechanism 112 may be housed in a second physical unit that adheres to the user's body, with wireless communication between the first physical unit and the second physical unit.

An expected bolus interval is an interval during which a bolus is expected to be taken. The expected bolus interval may be programmed (using user interface 113) by the user. For example, the user may enter, for a particular expected bolus interval, a start time and duration. The user may alternatively enter a start time and an end time for the expected bolus interval. An insulin pump that “learns” expect bolus intervals automatically establishes an expected bolus interval using a history of boluses taken by the user. For example, if the user normally takes a bolus between 6:30 am and 7:30 am on weekdays, the “learning” insulin pump 100 establishes a “weekday” expected bolus interval that begins at 6:30 am and ends at 7:30 am on weekdays.

An early bolus interval is associated with an expected bolus interval. The early bolus interval is an interval beginning at some time prior to a start of the expected bolus interval and ending at the start of the expected bolus interval. For example, the user may choose to enter a value for duration of the early bolus interval as being thirty minutes (using user interface 113, for example by entering the duration of the early bolus interval using keypad 104 or by wireless port 108 communication with a computer). The early bolus interval is the thirty minute interval immediately preceding the associated expected bolus interval.

FIG. 1c is a graphical depiction of three expected bolus intervals and their associated early bolus intervals for a one day time period. A first expected bolus interval (e.g., for breakfast) begins at 6:30 am and ends at 7:30 am. A first early bolus interval, associated with the first expected bolus interval begins at 6:00 am and ends at 6:30 am. A second expected bolus interval (e.g., for lunch) begins at 11:30 am and ends at 1:00 pm. A second early bolus interval, associated with the second expected bolus interval, begins at 11:00 am and ends at 11:30 am. A third expected bolus interval (e.g., for dinner) begins at 6:00 pm and ends at 7:00 pm. A third early bolus interval, associated with the third expected bolus interval, begins at 5:00 pm and ends at 6:00 pm. It will be noted that more than one expected bolus interval may exist; expected bolus intervals need not be all of the same duration; early bolus intervals need not all be of the same duration. Furthermore, there may not be an early bolus interval associated with an expected bolus interval (e.g., duration of a particular early bolus interval may be “zero”).

Boluses not occurring within an expected bolus interval or an early bolus interval are simply considered to cover a random snack, or perhaps a “correction bolus” to correct a blood glucose value higher than expected at the time of the “correction bolus”.

Having now clearly described relevant components of insulin pump 100 with reference to FIGS. 1a and 1b, and clearly defined terms “expected bolus interval” and “early bolus interval”, several embodiments of method 300 will be described in detail.

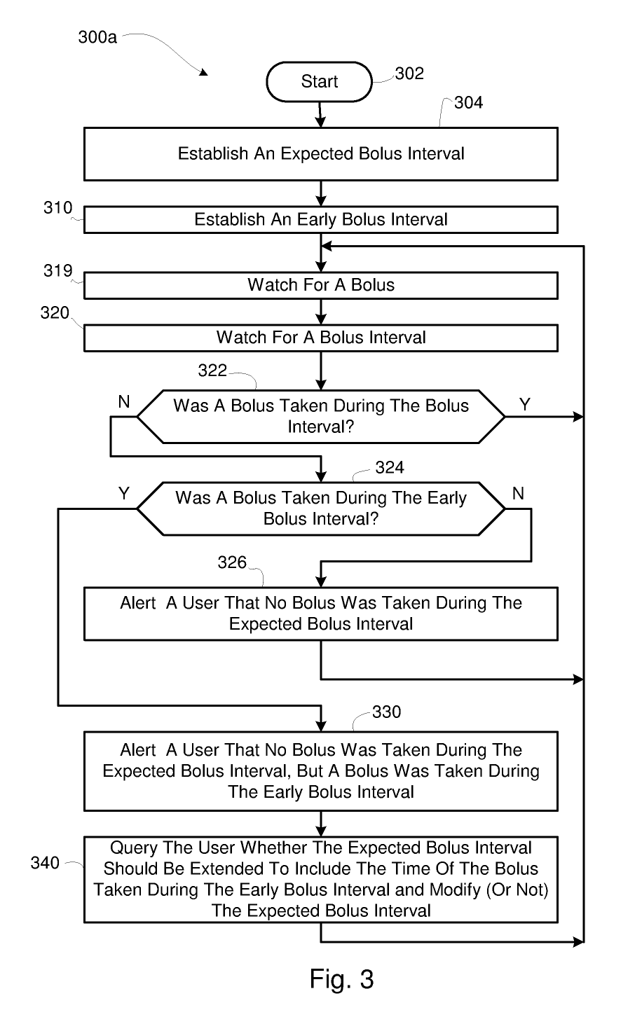

FIG. 3 is a high level flow chart of method 300a, a first embodiment of method 300. Method 300a alerts the user if no bolus was taken during an expected bolus interval, but that the user had taken a bolus shortly before the expected bolus interval (i.e., within the early bolus interval associated with the expected bolus interval).

Method 300a begins at step 302. In step 304, an expected bolus interval is established. As described earlier, the user may program the expected bolus interval (e.g., start time and duration; or start time and end time) using user interface 113. Alternatively, a “learning” insulin pump 100 is configured to “learn” the expected bolus interval from user history of taking boluses.

In step 310, an early bolus interval is established. For example, the user programs duration of the early bolus interval using user interface 113 (e.g., uses keypads 104 to, when prompted for a value for the early bolus interval, define a “thirty minutes” duration for the early bolus interval). Alternatively, a “learning” insulin pump 100 “learns” the expected bolus interval, and the “learning” pump also “learns” the early bolus interval. For example, if one hundred boluses are taken between 10:30 am and noon, and ninety five of those boluses are taken between 11:00 am and noon, insulin pump 100 determines that the expected bolus interval (when boluses are expected) begins at 11:00 am and ends at noon. However, the “learning” insulin pump 100 is also aware that the user occasionally takes a bolus between 10:30 am and 11:00 am (perhaps the user takes an early lunch on rare occasions), and “learning” insulin pump 100 establishes an early bolus interval between 10:30 am and 11:00 am.

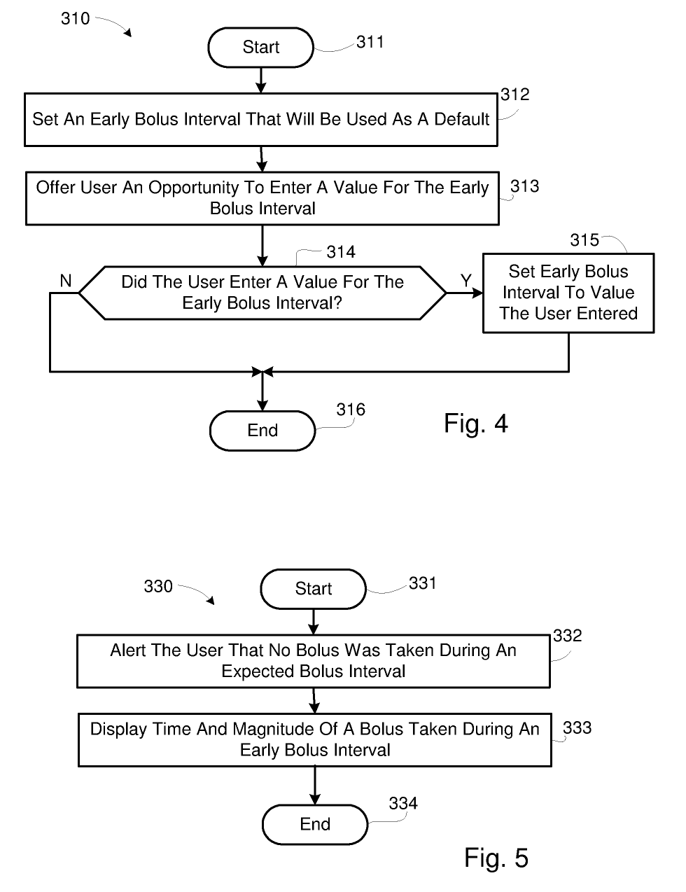

Step 310 is shown in more detail in FIG. 4. Step 311 begins the method of step 310. In step 312, a default early bolus interval is set. For example, a value of thirty minutes for a default early bolus interval may be stored in nonvolatile storage during manufacture of insulin pump 100. In step 313, insulin pump 100 offers the user an opportunity to enter duration for the early bolus interval that will be used instead of the default duration of the early bolus interval set in step 312. For example, processor 110, executing method 300 from memory 111, displays a message on display 102, “Change early bolus interval duration”, and display the default value of the early bolus interval, which was set in step 312. In response, the user may increase the duration by pressing key pad 104a one or more times, each press of key pad 104a telling processor 110 to increase the duration of the early bolus interval by one minute. Or, the user may decrease the duration by pressing keypad 104b one or more times, each press of key pad 104b telling processor 110 to decrease the duration of the early bolus interval by one minute. In step 314, if the user enters duration for the early bolus interval, the duration of the early bolus interval entered is set for use as the duration of the early bolus interval in step 315. If the user did not enter duration of the early bolus interval, the default set in step 312 is used as the duration of the early bolus interval. Step 316 ends the detailed method of step 310.

In step 319, insulin pump (processor 110, executing method 300a in memory 111) watches for a bolus being taken, and stores the time and magnitude of the bolus in memory 111 (or, alternatively, in a register (not shown) in processor 110).

In step 320 (FIG. 3), insulin pump 100 (processor 110, executing method 300a in memory 111) watches for an expected bolus interval, and, when finding the expected bolus interval further watches for a bolus being taken, as expected.

In step 322, at the end of the expected bolus interval, a check is made to see if a bolus was taken during the expected bolus interval. If a bolus was taken during the expected bolus interval, control passes to step 319 to watch for another bolus and expected bolus interval. If a bolus was not taken during the expected bolus interval, control passes to step 324. In step 324, a check is made to see if a bolus was taken during the early bolus interval associated with the expected bolus interval. If no bolus was taken during the early bolus interval, the user is alerted, in step 326, that no bolus was taken during the expected bolus interval. The user can then reset the alert if he or she did not eat during the expected bolus interval, and therefore did not need to take a bolus. However, if the user did eat, but forgot to take the required bolus, the user is reminded by the alert to take the required bolus. If step 324 determines that a bolus was taken during the early bolus interval, control passes to step 330.

In step 330, the user is alerted that no bolus was taken during the expected bolus interval, but that a bolus was taken during the early bolus interval. Alerting mechanism 114 alerts the user to check display 102, upon which is displayed, for example, a notice that no bolus was taken during the expected bolus interval, plus the time during the early bolus interval at which a bolus was taken, and the magnitude of that bolus. The user can then decide if a bolus is needed or not. For example, perhaps the user had a snack, and took a first bolus for the snack during the early bolus interval, and then did not eat during the expected bolus interval. No second bolus would be required. If, however, the user had the snack, and took a first bolus for the snack during the early bolus interval, and then had his or her meal during the expected bolus interval, a second bolus is required. In an embodiment, step 330 prevents the user from taking the second bolus until the user has confirmed that a second bolus is required.

FIG. 5 shows in additional detail of an exemplary method of step 330. Method 330 begins at step 331. In step 332 the user is alerted that no bolus was taken during an expected bolus interval. In step 333, the time and magnitude of a first bolus (taken during the early bolus interval) is shown on display 102. Once the alert has been reset, the user may take a second bolus, or not, as described above. Step 334 ends method 330.

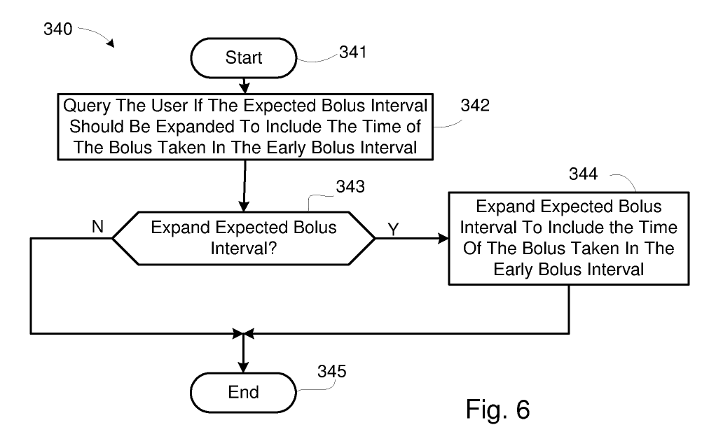

In step 340 of method 300a shown in FIG. 3, the user is queried whether the expected bolus interval should be extended to include the time of the bolus taken during the early bolus interval. For example, the user may have an expected bolus interval defined from 6:30 am to 7:30 am, but, because summer has arrived, has set his or her alarm clock to an earlier time and intends to regularly eat at 6:15 am. Step 340 is shown in more detail in exemplary method 340 shown in FIG. 6, which begins at step 340. In step 342, the user is queried if the expected bolus interval should be expanded to include the time of the bolus taken in the early bolus interval. If the user responds affirmatively in step 343, control passes to step 344, and the expected bolus interval is extended to include the time of the bolus taken during the early bolus interval. If the user does not respond affirmatively, the expected bolus interval is left unchanged. Method 340 of FIG. 6 ends at step 345.

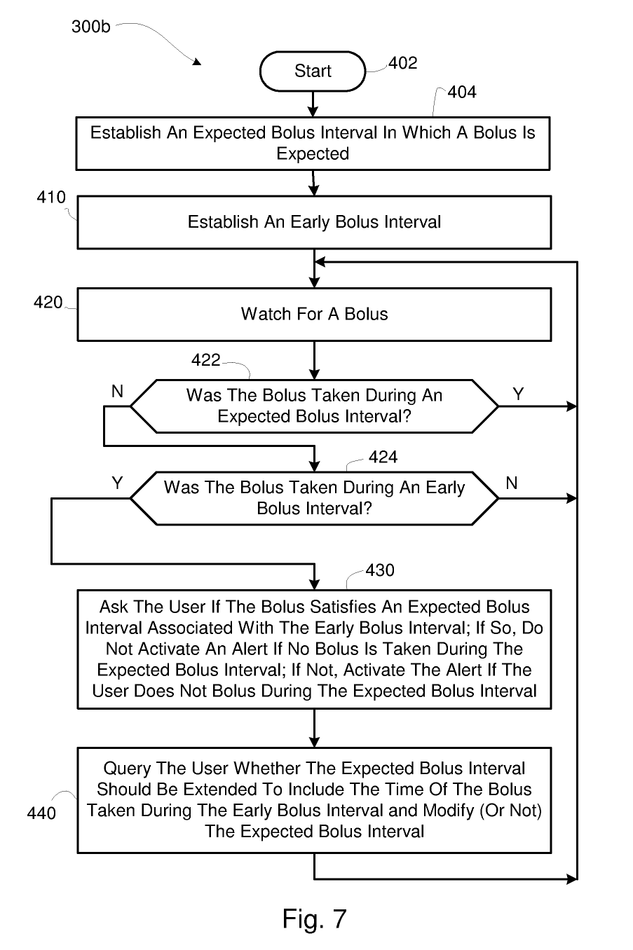

FIG. 7 shows a flowchart of a method 300b extension of method 300. Method 300b assumes a “missed meal bolus” method similar to method 300a is extant and configured to alert the user if no bolus is taken during an expected bolus interval. Method 300b watches for a bolus taken during an early bolus interval and, if the bolus is taken during the early bolus interval, asking the user if the bolus is to be interpreted as satisfying the associated expected bolus interval or not.

Method 300b begins at step 402. In step 404, an expected bolus interval is established, similarly to step 304 in method 300a. In step 410, an early bolus interval is established, similarly to step 310 of method 300a. In step 420, method 300b watches for a bolus. When a bolus is taken, control passes to step 422. Step 422 checks to see if the bolus was taken during an expected bolus interval; if so, step 422 ensures that no missed meal bolus alert will be issued at the end of the expected bolus interval; if not, step passes control to step 424.

Step 424 checks to see if the bolus was taken during an early bolus interval; if not, control passes to step 420, the bolus simply being assumed to be a “correction bolus” (a bolus taken to correct a high blood glucose condition, and which is not associated with an expected bolus interval or an early bolus interval), or that the bolus was taken with a snack eaten at a time outside of any expected bolus interval or early bolus interval. If step 424 determines that the bolus was taken during an early bolus interval, control passes to step 430.

Step 430 asks the user if the bolus is to be interpreted as satisfying an expected bolus interval associated with the early bolus interval. This would happen if the user simply ate and bolused a bit earlier than usual. If the user agrees, step 430 ensures that no missed meal bolus alert will be issued at the end of the associated expected bolus interval. If the user disagrees, an alert will be issued at the end of the expected bolus interval unless the user takes a bolus during the expected bolus interval. This would happen, for example, if the user has a snack shortly before the expected bolus interval and takes a first bolus for the snack, and then intends to eat a meal during the expected interval and take a bolus for what is eaten in the meal.

In step 440, the user is queried for whether he or she wishes to extend the expected bolus interval to the time of the bolus taken during the early bolus interval. If the user agrees, the expected bolus interval start time is moved to the time of the bolus taken during the early bolus interval. The user may agree if he or she knows that normal meal times have changed to an earlier time. Step 440 may also move the ending time of the expected bolus interval earlier by the same amount of time that the start time of the expected bolus interval was moved, so that the duration of the expected bolus interval remains constant. Step 440 may move a start time of the early bolus interval earlier by the same amount that the start time of the expected bolus interval was moved such that the duration of the early bolus interval remains constant. If the user disagrees with the query in step 440, step 440 takes no action with regard to moving the start time or the stop time of the expected bolus interval.

What is claimed is:

1. A insulin pump comprising:
a timer;
an alerting mechanism;
an expected bolus interval; and
an early bolus interval, an ending time of the early bolus interval being the same time as a starting time of the expected bolus interval;
wherein the insulin pump is configured to detect a bolus taken during the early bolus interval, and use the determination of the bolus taken during the early bolus interval in determination of whether to activate the alerting mechanism if no bolus is taken during the expected bolus interval.

2. The insulin pump of claim 1, further comprising a default value for duration of the early bolus interval.

3. The insulin pump of claim 2, further comprising a user interface configured to receive a user-programmable value for the duration of the early bolus interval, the user-programmable value for the duration of the early bolus interval, if received, used by the insulin pump instead of the default value for duration of the early bolus interval.

4. The insulin pump of claim 3, wherein the user interface receives the user-programmable value for duration of the early bolus interval from a key pad.

5. The insulin pump of claim 3, wherein the user interface receives the user-programmable value for duration of the early bolus interval from a wireless port.

6. The insulin pump of claim 1 configured to, when detecting a bolus taken during the early bolus interval, queries a user of the insulin pump whether the bolus taken during the early bolus interval satisfies the associated expected bolus interval; if so, no alert is issued if a bolus is not taken during the associated expected bolus interval.

7. The insulin pump of claim 6 further configured to receive direction from the user to extend the expected bolus interval to include a time at which the bolus taken during the early bolus interval was delivered.

8. The insulin pump of claim 1 configured to, when a bolus is detected during an early bolus interval, and when no bolus is detected during the associated expected bolus interval, alerts the user that a bolus was detected during the early bolus interval and that no bolus was taken during the associated expected bolus interval.

9. The insulin pump of claim 8 further configured to receive input via the user interface as to whether to take a bolus.

10. The insulin pump of claim 8 further configured to receive direction from the user to extend the expected bolus interval to include a time at which the bolus taken during the early bolus interval was delivered.

11. A method executed in an insulin pump having a missed meal bolus alert to reduce likelihood of a user taking an unwanted second bolus comprising the steps of:
establishing an expected bolus interval during which a bolus is expected to be taken by the user;
establishing an early bolus interval immediately preceding the expected bolus interval; and
if a bolus was not taken during the expected bolus interval, but a bolus was taken during the early bolus interval, alerting the user that no bolus was taken during the expected bolus interval but a bolus was taken during the early bolus interval.

12. The method of claim 11, further comprising a step of not allowing the user to take a bolus until the user has responded to the alert that no bolus was taken during the expected bolus interval but a bolus was taken during the early bolus interval.

13. The method of claim 11, further comprising the steps of:
querying the user whether the expected bolus interval should be expanded to include a time that the bolus taken during the early bolus interval was taken;
if the user responds affirmatively to the querying, a starting time of the expected bolus interval is changed to include the time that the bolus taken during the early bolus interval was taken; and
if the user responds negatively to the querying, the expected bolus interval is not changed.

14. A method executed in an insulin pump having a missed meal bolus alert to reduce likelihood of a user taking an unwanted second bolus comprising the steps of:
establishing an expected bolus interval during which a bolus is expected to be taken by the user;
establishing an early bolus interval immediately preceding the expected bolus interval; and
if a bolus is taken during the early bolus interval, asking the user if the bolus taken during the early bolus interval satisfies an expected bolus interval associated with the early bolus interval;
if the user responds affirmatively, not activating an alert if the user does not take a bolus during the associated expected bolus interval; and
if the user responds negatively, activating the alert if the user does not take a bolus during the associated expected bolus interval.

15. The method of claim 14, further comprising the steps of:
querying the user whether the expected bolus interval should be expanded to include a time that the bolus taken during the early bolus interval was taken;
if the user responds affirmatively to the querying, a starting time of the expected bolus interval is changed to include the time that the bolus taken during the early bolus interval was taken; and
if the user responds negatively to the querying, the expected bolus interval is not changed.

16. The method of claim 15, further comprising a step of, if the starting time of the expected bolus interval is changed to include the time that the bolus taken during the early bolus interval, changing and ending time of the expected bolus interval such that a duration of the expected bolus interval remains constant.
